(19) United States
(12) Patent Application Publication
(10) Pub. No.: US 2008/0306430 A1
(43) Pub. Date: Dec. 11, 2008

(54) Method and device for the insertion of intra-vaginal sponges

(76) Inventor: Alon Avisar, Herzlia (IL)

Correspondence Address: UZI EZRA HAVOSHA & PARTNERS, HADAR-DAFNA HOUSE 39 SHAOUL HAMELECH STREET, TEL AVIV, 64928, IL

(21) Appl. No.: 11/811,296

(22) Filed: Jun. 11, 2007

(51) Int. Cl. A61F 13/20; A61M 1/00; A61M 31/00

(52) U.S. Cl. 604/15; 604/28; 604/514

(57) ABSTRACT

Magazine of sponges used for insertion into the vagina of mammalian females and an applicator to facilitate the said insertion.

### FIELD of the INVENTION

This invention is in the field of veterinary intra-vaginal insertions and more specifically relates to method of using an applicator and magazines for the insertion of intra-vaginal sponges into the vagina of a mammalian female.

### BACKGROUND of the INVENTION

This invention relates to the administration of medicaments and the like and particularly to the administration of pro-gestational compounds to mammals. Specifically, this invention relates to the application of sponges which are impregnated with a pro-gestational compound and which are suitable for intra-vaginal administration. The sponges have special importance in connection with the control of fertility, especially in domestic animals, by the control of ovulation and estrus. The sponge is usually, but not limited to, a cylindrical shape and it is tied to a cord. After the sponge is inserted into the vagina, part of the cord remains out of the vagina.

The sponge releases pro-gestational compounds systemically for a desired period of time. At the end of that time period the sponge is removed by pulling the cord and then the whole sponge is taken out of the body. This is a process that is known in the art revealed in U.S. Pat. No. 3,916,898 issued to Robinson.

Two main methods of insertion into the vagina of the animals have been used till now.

The first is a duckbilled speculum. The vagina is dilated and the sponge is pushed as far as possible into the anterior vagina with a plastic plunger. The second method employs a simple applicator—a tapered plastic tube angle cut at the insertion end and slightly belled out at the other. A plunger of similar material is used to force a sponge down the tube which is then inserted as far as possible into the vagina. The sponge is gently forced out with the plunger. In both methods the speculum (or applicator) and plunger are withdrawn leaving the sponge in place.

In the past applicators employed for this purpose, have been known to provide for the delivery of a single sponge at each loading. When there are a large group of animals (e.g. sheep and goats) the method is very slow and tedious. Further more the method is not sterile and may cause vaginitis or endometritis. When the loading is done manually, there is a high risk for infection from a contaminated sponge.

This invention relates to a new method of loading and packing of the sponges in a magazine. The use of such magazines and suitable applicator will facilitate and speed up the operation and will reduce the likelihood of infections in the genital tract.

### SUMMARY of the INVENTION

It is to be understood that both the foregoing general description and the following detailed description present embodiments of the invention, and are intended to provide an overview, or framework, for understanding the nature and character of the invention as it is claimed. The accompanying drawings are included to provide a further understanding of the invention, and are incorporated into and constitute a part of this specification. The drawings illustrate various embodiments of the invention and, together with the description serve to explain the principles and operations of the invention.

This invention relates to methods of packing, loading, and inserting of intra-vaginal sponges.

According to the invention the new, improved method includes an applicator and magazines for the sponges. The method of use is by loading the sponge from a magazine to an applicator and from the applicator to the vagina of the animal.

The sponges would be packed in special design magazines with an aseptic technique.

The loading of the magazine into the applicator is done by pushing up the magazine in the handle of the applicator. The internal diameter of the said handle could be slightly smaller than the diameter of the sponges. This would ensure that the sponges would hold themselves in the said handle by virtue of the pressure of the sponge against the internal walls of the said handle and without other mechanisms to hold the sponges in place. The handle of the applicator also serves as a magazine container.

Before the loading of the magazine the applicator plunger should be pulled out as far as possible. The distal end of the plunger is round and larger then the diameter of the plunger. When the plunger is pulled out through the hole in the handle it remains attached to the handle because of the large distal end. This large end will also facilitate the plunger's main purpose namely to push the sponge out of the applicator. The larger the area of the plunger end that presses on the sponge the easier the plunger will push the sponge.

After the magazine is in place, the speculum (the exit from the applicator) is inserted into the vagina. A single sponge can be loaded to the speculum of the applicator by pushing the plunger. Then the sponge is forced down the tube shape of the speculum by pushing the plunger farther until the sponge is forced out into the vagina. The applicator is then withdrawn leaving the sponge in place and the string of the sponge hanging out of the animal.

The speculum part could be washed and disinfected by dipping only the speculum part in a suitable aqueous solution. Next step is to pull the magazine with new sponge up into the right place, namely so the next sponge is opposite the plunger end when the plunger is pulled back. Then in the same way loading and forcing out a new sponge with the plunger. Cleaning and repeating the same procedure between one animal to the other.

### BRIEF DESCRIPTION of the DRAWINGS

The accompanying drawings, which are incorporated in and form a part of this specification, illustrate embodiments of the invention and, together with the description, serve to explain, by way of example only, the principles of the invention:

FIG. 1 is a schematic depiction of the applicator.

FIG. 2 is a schematic depiction of a column of sponges used with this invention.

FIG. 3 is a schematic depiction of a magazine to hold the sponges.

FIG. 4 is a schematic depiction of the sponge applicator in its various stages of action.

### DETAILED DESCRIPTION of the PREFERRED EMBODIMENTS

As will be appreciated the present invention is capable of other and different embodiments than those discussed above and described in more detail below, and its several details are capable of modifications in various aspects, all without departing from the spirit of the invention. Accordingly, the drawings and description of the embodiments set forth below are to be regarded as illustrative in nature and not restrictive.

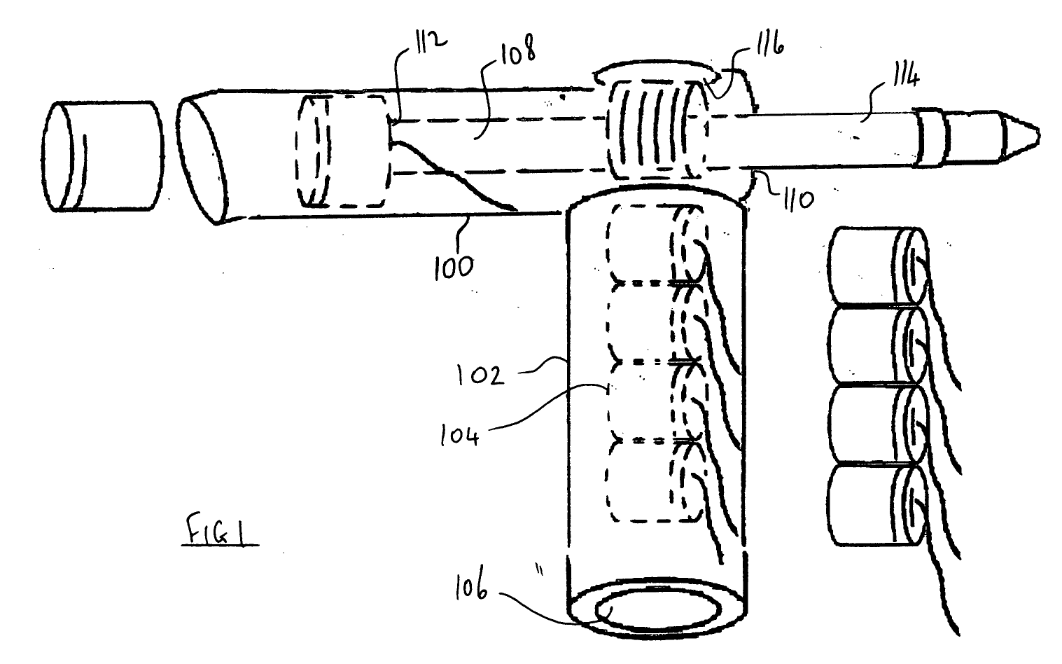

FIG. 1 shows an applicator 100 of this invention. It has a handle 102 that has another function namely to house the column or magazine 104 of sponges, similar to a magazine of bullets that is inserted into the handle of a pistol. The insertion of the sponges or the magazine 104 of sponges is from a hole 106 in the base of the handle 102. The internal diameter of the handle 102 could be slightly smaller than the diameter of the sponges in the magazine of sponges 104 so that the sponges are held in the handle 102 by means of their own force pressing against the internal walls of the handle 102.

The sponges are pushed forward and out of the applicator by means of the plunger 108. Plunger 108 has a handle 114 and at its distal end 112 is slightly enlarged. This enlargement serves at least two purposes namely, to help push out the sponge without damaging it and secondly to prevent the plunger from exiting from the hole 110 at the back of the applicator where the said hole 110 is big enough for the plunger 108 to slide back and forth freely and small enough to prevent the plunger from exiting from the hole 110. The handle 114 is to assist the manual operation of the plunger.

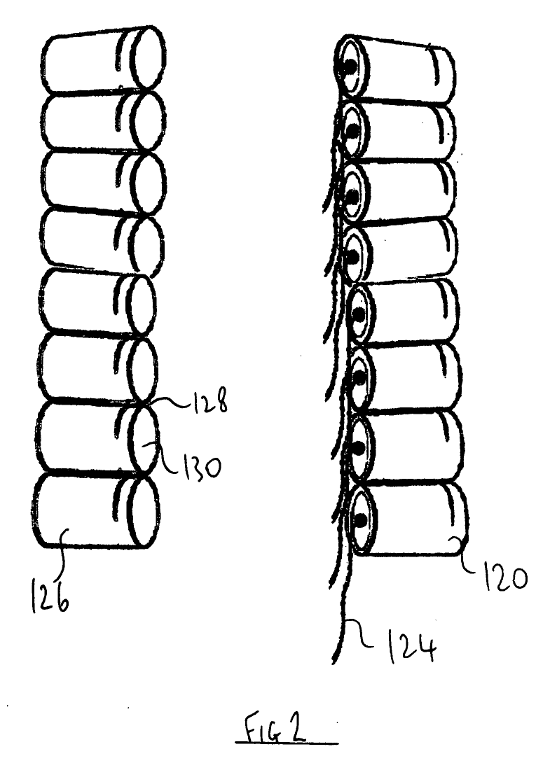

The hole 116 is for the empty magazine container FIG. 2 124 to exit from the applicator after the sponge has been pushed out of its compartment. Optionally the hole 116 can be used for the insertion of individual sponges into the applicator.

FIG. 2 shows the sponges 120 in a column as they would be stacked in a magazine 126. The string 124 attached to each sponge is for the purpose of retrieving the sponge from inside the animal after sufficient time has passed for the substances in the sponge to dissipate inside the animal. The magazine 126 that holds the sponges is seen here from the side view. They could be made from any suitable material for example, nylon or plastic. They are large enough to hold the sponge sufficiently tightly that the sponge does not fall out when the magazine 126 is moved around, removed from its storage and placed into the handle of the applicator FIG. 1 102. However, the sponge is not so tightly held by the magazine 126 that it is hard to push out of the magazine 126 by the plunger end FIG. 1 112.

The individual magazine cylinders 130 are joined tangently 128 along their length, whereby all the sponges in one magazine remain attached while at the same time being flexible.

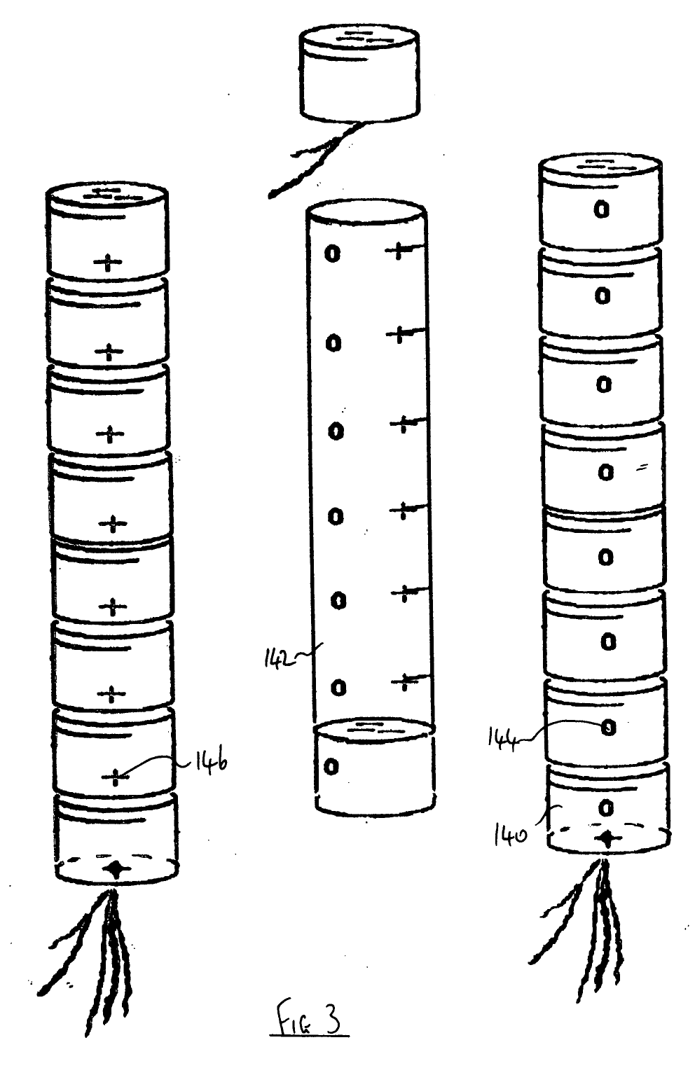

FIG. 3 shows two sides of an alternative embodiment of magazine to hold and dispense sponges. In this embodiment the sponges 140 stand vertically, each cylindrical sponge's flat circular side lying on the flat circular side of its neighbor as opposed to the position of the sponges described in FIG. 2 that lie sideways touching tangently. The plastic sleeve 142 holding the sponges 140 have a hole 144 opposite each sponge 140 on one side of the sleeve 142 and a cross-split 146 on the opposite side of the sleeve 142. The hole 144 and split 146 are to facilitate the plunger FIG. 1 108 to enter the hole 144 and push out the sponge 140 through the split 146.

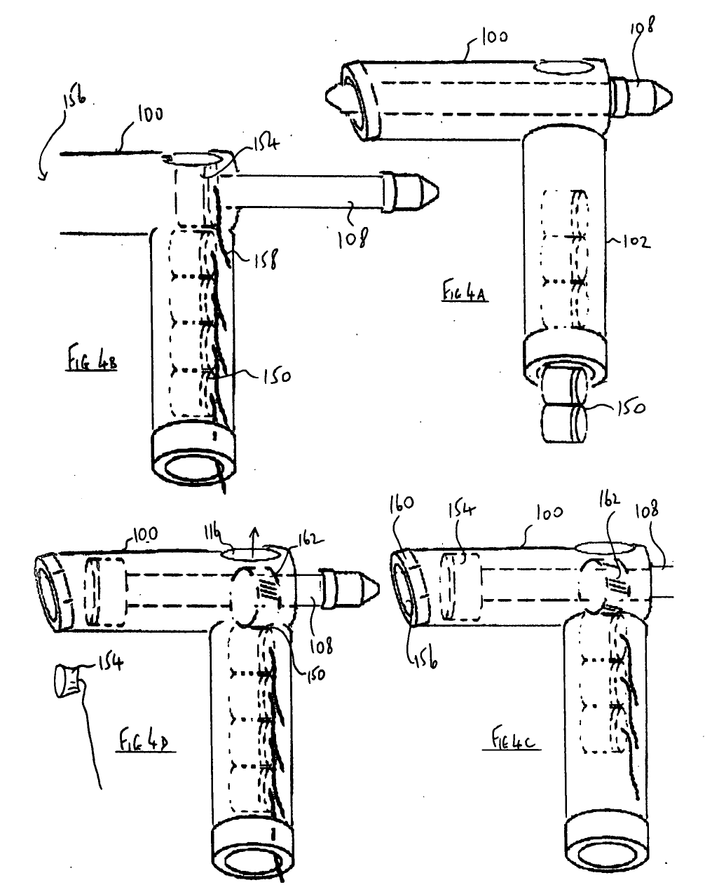

FIG. 4A shows a magazine of sponges 150 being pushed up into the handle 102 of an applicator 100 of this invention. The plunger 108 is pulled back to leave the space free for the magazine 150 of sponges to move into place and to be positioned correctly to push the top sponge out through the front of the applicator 100.

FIG. 4B shows the magazine 150 of sponges in place and the plunger 108 positioned to push the top sponge 154 towards the speculum opening 156 of the applicator 100. The sponges in the magazine 150 each have a string 158 attached which is eventually used to pull the sponge out of the animal's body.

FIG. 4C shows the plunger 108 pushing the top sponge 154 of the magazine 150 of sponges out from the magazine 150 into the nozzle or speculum 160 of the applicator 100 towards the opening 156. At this stage the speculum 160 would be inserted into the animal's vagina so that it is in position before pushing the sponge 154 out of the speculum 160 part of the applicator 100 and into the animal.

After the sponge 154 has been pushed out of the magazine 150 there remains an empty magazine container 162.

FIG. 4D shows the plunger 108 having pushed the sponge 154 out of the applicator 100 and is ready to be pulled back to be out of the way of the magazine 150 of sponges. The empty magazine container 162 can be manually pulled from its position up through the hole 116 in the applicator 100. As the magazine 150 is pulled up the next sponge is brought into position opposite plunger 108 ready for that sponge to be pushed out, and so on.

The present invention is not intended to be limited to the embodiments described above, but to encompass any and all embodiments within the scope of the following claims.

What is claimed:

1. A method and device for the insertion of intra-vaginal sponges with the aid of an applicator comprising;
a) a hollow handle for inserting a magazine of intra-vaginal sponges,
b) a tapered speculum-type nozzle for insertion into a vagina having an exit hole for the said sponge to enter into the said vagina,
c) a plunger for pushing the said sponge out of the said exit hole into the said vagina, and
d) a magazine of the said sponges to fit into the said handle,
whereby the said sponges can be speedily, conveniently and hygienically inserted in the vaginas of female animals and whereby there is a means to hold the said sponges in place ready for the said insertion and until needed for use.

2. A method and device for the insertion of intra-vaginal sponges with the aid of an applicator as claimed in claim 1 wherein the said magazine is a series of open ended cylindrical containers each being a size for cylindrical shaped sponges to fit in firmly.

3. A method and device for the insertion of intra-vaginal sponges with the aid of an applicator as claimed in claim 1 wherein the said magazine is a series of open ended cylindrical containers joined together along the length of each cylinder tangently one cylinder one top of its neighbor with the axis of the length of the said cylinder being at right-angles to the line of joined cylinders whereby the said sponges lie on their circular sides with the flat end of the said sponge facing forward and backward when the magazine of sponges is held vertically.

4. A method and device for the insertion of intra-vaginal sponges with the aid of an applicator as claimed in claim 1 wherein the said magazine is a cylindrical shaped container with at least one slit or hole in the side wall of the said container at intervals to match the size of the said sponges and at least one slit or hole equidistant to the first set of said slits or hole on the opposite side of the said cylindrical shaped container thereby enabling each sponge stacked into the said cylindrical container magazine to be pushed out of the slit or hole adjacent to each sponge.

5. A method and device for the insertion of intra-vaginal sponges with the aid of an applicator as claimed in claim 1 wherein the said sponges may be shaped in any one or more of the following shapes namely cylindrical, oval, spherical, circular or any convenient shape for the herein intended and stated use of the said sponges.

6. A method and device for the insertion of intra-vaginal sponges with the aid of an applicator as claimed in claim 5 wherein container of the said sponges is shaped accordingly to hold the sponges firmly in place until pushed out into the animal's vagina.

7. A method and device for the insertion of intra-vaginal sponges with the aid of an applicator as claimed in claim 1 wherein the said applicator has a plunger for pushing the said sponges out of the said nozzle of the applicator where the said plunger has at its distal end from the handle, a smooth and widened end thereby enabling the said plunger to push the said sponges without damaging them and to prevent the said plunger from being detached from the applicator.

8. A method and device for holding together intra-vaginal sponges comprising a magazine to hold at least two of the said sponges separated one sponge from its neighbor by individual sponge containers open at its back and front side whereby there is a means to store the said sponges in an orderly manner and whereby the said sponges are held in place ready for the said insertion.

9. A method and device for holding together intra-vaginal sponges as claimed in claim 8 wherein the said magazine is a series of open ended cylindrical containers each being a size for a cylindrical shaped sponge to fit in firmly.

10. A method and device for holding together intra-vaginal sponges as claimed in claim 8 wherein the said magazine is a series of open ended cylindrical containers joined together along the length of each cylinder tangently one cylinder one top of its neighbor with the axis of the length of the said cylinder being at right-angles to the line of joined cylinders whereby the said sponges lie on their circular sides with the flat end of the said sponge facing forward and backward when the magazine of sponges is held vertically.

11. A method and device holding together intra-vaginal sponges as claimed in claim 8 wherein the said magazine is a cylindrical shaped container with at least one slit or hole in the side wall of the said container at intervals to match the size of the said sponges and at least one slit or hole equidistant to the first set of said slits or hole on the opposite side of the said cylindrical shaped container thereby enabling each sponge stacked into the said cylindrical container magazine to be pushed out of the slit or hole adjacent to each sponge.

12. A method and device for holding together intra-vaginal sponges as claimed in claim 8 wherein the said sponges may be shaped in any one or more of the following shapes namely cylindrical, oval, spherical, circular or any convenient shape for the herein intended and stated use of the said sponges.

13. A method and device for holding together intra-vaginal sponges as claimed in claim 12 wherein container of the said sponges is shaped accordingly to hold the sponges firmly in place until pushed out into the animal's vagina.

14. A method and device for the insertion of intra-vaginal sponges comprising;
a) an applicator with a hollow handle for inserting a magazine of intra-vaginal sponges,
b) a tapered speculum-type nozzle for insertion into a vagina having an exit hole for the said sponge to enter into the said vagina, and
c) a plunger for pushing the said sponge out of the said applicator into the said vagina,
whereby the said sponges can be speedily, conveniently and hygienically inserted in the vaginas of female animals.

15. A method and device for the insertion of intra-vaginal sponges as claimed in claim 14 wherein the said applicator has a plunger for pushing the said sponges out of the said nozzle of the applicator where the said plunger has at its distal end from the handle, a smooth and widened end thereby enabling the said plunger to push the said sponges without damaging them and to prevent the said plunger from being detached from the applicator.

16. A method and device for the insertion of intra-vaginal sponges as claimed in claim 14 further comprising a hole in the upper end of the said applicator whereby spent sponge containers can be extracted from the said applicator and the next sponge in its container be pulled into the position for pushing out of the said applicator with the aid of the said plunger.
